(19) United States
(12) Patent Application Publication
(10) Pub. No.: US 2022/0095923 A1
(43) Pub. Date: Mar. 31, 2022

(54) SYSTEM AND APPARATUS FOR DETECTING DISEASES

(71) Applicant: Techara LLC, Concord, NH (US)

(72) Inventors: Karleen Seybold, Concord, NH (US); Nikhil Dhinagar, Los Angeles, CA (US)

(21) Appl. No.: 17/489,400

(22) Filed: Sep. 29, 2021

Related U.S. Application Data
(60) Provisional application No. 63/084,982, filed on Sep. 29, 2020.

(51) Int. Cl. A61B 5/00; A61B 5/08

(52) U.S. Cl. CPC A61B 5/0075; A61B 5/443; A61B 5/082; A61B 5/0064

(57) ABSTRACT

A system and apparatus for detecting diseases that combines biosensors and skin imaging. The system and apparatus combines bio-sensing technology for measuring volatile organic compounds (VOCs) emanating from the breath, oral cavity, and/or skin with multispectral high-resolution imaging of the skin utilizing visible and infrared wavelengths to detect specific diseases and/or medical conditions.

### CROSS-REFERENCE TO RELATED APPLICATIONS

This patent application claims priority to provisional patent application having Ser. No. 63/084,982, filed Sep. 29, 2020, which is herein incorporated by reference in its entirety.

### FIELD OF INVENTION

The present invention is directed to a system and apparatus that combines oral cavity sensing and multi-spectral high-resolution imaging to accurately and quickly screen for or detect diseases and/or disease states of individuals. More particularly, the present invention includes a system and apparatus that combines bio-sensing technology for measuring volatile organic compounds (VOCs) emanating from the breath, oral cavity; and/or skin with multispectral high-resolution imaging of the skin utilizing visible and infrared wavelengths to detect specific diseases and/or medical conditions. Specific diseases and/or medical conditions are detected by utilizing identifying signatures that are determined by linking the bio-sensing data and imaging data and integrating artificial intelligence (AI) algorithms to fine tune the correlation. The present invention is especially applicable in screening/detecting of COVID-19 before the onset of visible symptoms.

The present invention is also directed to a method for creating a system and apparatus for performing rapid, accurate, non-invasive point-of-care tests for detecting various diseases and medical conditions which includes the steps of utilizing bio-sensing technology to measure VOCs from the breath, oral cavity, and/or skin of individuals with a known VOC disease bio-marker and healthy/non-diseased individuals, utilizing high resolution multispectral imaging to obtain skin images of the same individuals with a known VOC disease bio-marker and healthy/non-diseased individuals, processing the data obtained from measuring VOCs and imaging the skin to determine/identify the infrared wave signature correlation for the disease that matches the VOCs, and integrating AI to develop a filter or detector for the infrared wave signature. The filter or detector for the infrared wave signature can then be utilized in a point-of-care test to identify diseased and non-diseased individuals. The point-of-care test may be performed with an imaging booth or a hand-held smart device.

### BACKGROUND OF THE INVENTION

Many of the currently existing methods and apparatus for detecting various diseases and medical conditions are invasive, slow, imprecise, and/or require laboratory agents for processing. For example, many current systems and methods require blood draws or the introduction of instruments or other objects into the body. Further, many current systems and methods cannot provide accurate and real-time results for detecting diseases and medical conditions. The present invention provides a system and apparatus for detecting diseases and medical conditions that is non-invasive, accurate, performed in real-time without prolonged lag times, and which is not dependent on laboratory agents.

The present invention is particularly capable of providing a system and apparatus for point-of-care early screening and detection of rapidly spreading communicable diseases such as COVID-19. It is already known that volatile organic compounds (VOCs) emitted form biological waste (such as breath, skin, stool, or urine) can be used to diagnose and monitor a range of medical conditions. VOCs are molecules with high vapor pressure that contain at least one carbon and hydrogen molecule. They are continuously being emitted from the body and can provide insight into the health and well-being of an individual.

Skin has a spectral nature, so an entirely different picture presents itself when combining visible imaging and moving into the short-wave infrared wavelength range (780 to 1400 nm). Infrared imaging at various wavelengths makes it possible to visualize material properties such as water content, temperature, or chemical components which cannot be identified with conventional surface inspection. Scientists at the Bonn-Rhein-Sieg University of Applied Sciences developed the “spectral signature” method within the framework of their research project on face detection using face biometrics. Journal of Sensors Article ID 456368: Design of an Active Multispectral SWIR Camera System for Skin Detection and Face Verification; Holger Steiner et al.; March 2015. In this method, the albedo of skin in a wavelength range between 900 and 1500 nm was analyzed and a skin-specific value range was determined. At approximately 1450 M, the characteristic absorption characteristics of skin, independent of skin type, are particularly large. Above 900 nm, skin pigments have no relevant influence on albedo, thus absorption by the water contained in the skin increasingly gains in influence. The method of facial recognition investigation was implemented with an active camera system in which an infrared camera is mounted in the middle of a ring of LEDs Three rows of LEDs transmit light in different wavelengths within the defined range onto the face to be analyzed, whereby the measurement becomes even more reliable. The camera records the reflected short-wave infrared light.

Current Covid-19 screening suffers from a number of key drawbacks including false negative results, specific lab reagent requirements, prolonged lag time to results and the inability to detect early signs of the disease i.e., asymptomatic patients. COVID-19 is a respiratory condition that affects the lungs, but also the gut (the Covid virus can be found in the gut even after the individual has recovered from the main infection). This will result in alterations in metabolic function, gastro function, inflammation and host response in fighting the infection, all of which can be detected in breath and other bio-waste emitted through the skin. There is support from a previous study showing that VOCs (volatile organic compounds) such as ethyl butanoate, acetone and isopropanol-1 emission correlate specifically with COVID-19, with these chemicals being clear metabolic and inflammation bio-markers. H. Chen, X. Qi, J. Ma, C. Zhang, H. Few, and M. Yao, “Breath-borne VOC Biomarkers CVID-19,” medRxiv, p. 2020.06.21.20136523, 2020. High temperature (fever) is a known bio-marker to COVID-19 with associated metabolic changes with resultant changes to skin (tone).

There is a need for rapid, accurate, non-invasive point-of-care tests for detecting various diseases and medical conditions. In addition, there is an urgent need for a rapid, accurate, non-invasive point-of-care test for COVID-19 that can be carried out in medical settings, airports, shopping malls and factories that can identify those without the disease. There is also a need for a rapid, accurate, non-invasive point-of-care test for those already identified as COVID-19 positive to determine those who are likely to require additional medical interventions such as mechanical ventilation or intensive care.

The present invention provides a system and apparatus that combines cutting edge bio-sensing and multi spectral high-resolution imaging to detect specific diseases and/or medical conditions and is particularly suited to detect rapidly spreading communicable diseases such as COVID-19. Specific diseases and/or medical conditions, such as COVID-19, are detected by utilizing identifying signatures that are determined by linking the bio-sensing data and imaging data.

### SUMMARY OF THE INVENTION

The present invention is directed to a system and apparatus for performing rapid, accurate, non-invasive point-of-care tests for detecting various diseases and medical conditions that includes both bio-sensing technology for measuring volatile organic compounds (VOCs) emanating from the breath, oral cavity, and/or skin with multispectral high-resolution imaging of the skin utilizing visible and infrared wavelengths. Specific diseases and/or medical conditions are detected by utilizing identifying signatures that are determined by linking and correlating the bio-sensing data and imaging data using AI methodologies and algorithms.

In one exemplary embodiment, the system and apparatus of the present invention for detecting various diseases and medical conditions includes an imaging booth capable of performing full body high resolution multispectral imaging of an individual and a Gas Chromatograph (GC) and an Ion Mobility Spectrometer instrument with a mouthpiece for receiving an individual's breath that is capable of chemically analyzing an individual's breath for VOCs (such as the G.A.S. BREATHSPEC). The mouthpiece or mouth section of the instrument extends into the imaging booth so that an individual undergoing a full body imaging session utilizing the imaging booth can also provide a breath sample while undergoing full body imaging.

In another exemplary embodiment, the system and apparatus of the present invention for detecting various diseases and medical conditions includes an imaging booth capable of performing full body high resolution multispectral imaging of an individual and a Gas Chromatograph (GC) and an Ion Mobility Spectrometer instrument which is used separately, apart from the imaging booth, to receive an individual's breath and chemically analyzing the individual's breath for VOCs (such as the G.A.S. BREATHSPEC).

In still another exemplary embodiment, the system and apparatus of the present invention for detecting various diseases and medical conditions includes a handheld smart device for remote patient screening and disease detecting that incorporates portable sensors or filters for measuring VOCs and imaging skin.

In yet another exemplary embodiment, the system and apparatus of the present invention is specifically designed to detect or screen for COVID-19 and includes an imaging booth capable of performing full body high resolution multispectral imaging of an individual and a Gas Chromatograph (GC) and an Ion Mobility Spectrometer instrument with a mouthpiece for receiving an individual's breath that is capable of chemically analyzing an individual's breath for VOCs (such as the G.A.S. BREATHSPEC). The mouthpiece or mouth section of the instrument extends into the imaging booth so that an individual undergoing a full body imaging session utilizing the imaging booth can also provide a breath sample while undergoing full body imaging. The system and apparatus includes a filter or detector for detecting a specific infrared wave signature or signatures that correlates COVID-19 with measured VOCs and skin imaging of individuals known to have COVID-19.

In an alternative embodiment, the system and apparatus specifically designed to detect or screen for COVID-19 may comprise an imaging booth for skin imaging and a Gas Chromatograph (GC) and an Ion Mobility Spectrometer instrument that is separate from the imaging booth. In yet another alternative embodiment, the system and apparatus specifically designed to detect or screen for COVID-19 may comprise a handheld smart device for remote patient screening and detecting of COVID-19 that incorporates portable sensors or filters for measuring VOCs and imaging skin. All embodiments of the system and apparatus for detecting or screening for COVID-19 would include a filter or detector for detecting a specific infrared wave signature or signatures that correlates COVID-19 with measured VOCs and skin imaging of individuals known to have COVID-19.

The present invention is also directed to a method for creating a system and apparatus for performing rapid, accurate, non-invasive point-of-care tests for detecting various diseases and medical conditions which includes the steps of utilizing bio-sensing technology to measure VOCs from the breath, oral cavity, and/or skin of individuals with a known VOC disease bio-marker and healthy/non-diseased individuals, utilizing high resolution multispectral imaging to obtain skin images of the same individuals with a known VOC disease bio-marker and healthy/non-diseased individuals, processing the data obtained from measuring VOCs and imaging the skin to determine/identify the infrared wave signature for the disease that matches the VOCs, and developing a filter or detector for the infrared wave signature. The filter or detector for the infrared wave signature can then be utilized in a point-of-care test to identify diseased and non-diseased individuals. The point-of-care test may be performed with an imaging booth or a hand-held smart device.

The present invention is also directed to a method for creating a system and apparatus for performing rapid, accurate, non-invasive point-of-care tests for detecting COVID-19 which includes the steps of utilizing bio-sensing technology to measure VOCs from the breath, oral cavity, and/or skin of individuals with a known COVID-19 VOC bio-marker and healthy/non-diseased individuals, utilizing high resolution multispectral imaging to obtain skin images of the same individuals with a known COVID-19 VOC bio-marker and healthy/non-diseased individuals, processing the data obtained from measuring VOCs and imaging the skin to determine/identify the infrared wave signature for COVID-19 that matches and correlates the VOCs, integrating AI and developing a filter or detector for the infrared wave signature for COVID-19. The filter or detector for the infrared wave signature for COVID-19 can then be utilized in a point-of-care test to identify those individuals with and without COVID-19. The point-of-care test may be performed with an imaging booth or a hand-held smart device.

The present invention is further directed to a method for creating identifying signatures or detectors for specific diseases and/or medical conditions that combines biosensing technology to measure volatile organic compounds emanating from diseased and non-diseased individuals and high resolution multispectral skin imaging of diseased and non-diseased individuals to obtain data that can be processed to find correlations in the data that enable the determination of an infrared wave signature for specific diseases and/or medical conditions. Once an infrared wave identifying signature or detector is determined for a specific disease or medical condition, it can be used in a program application that is part of an imaging booth (such as the exemplary imaging booth shown in FIG. 3) that obtains and processes multiple high resolution skin images of an individual to determine whether or not the individual has the specific disease or medical condition. Once the infrared wave identifying signature for a specific disease is determined, the presence of the disease in an in individual may be determined via skin imaging and processing of the skin images without the use of a gas chromatograph and ion mobility spectrometer. The present invention also includes the use of multiple infrared wave identifying signatures for multiple different diseases in the program application that is part of the skim imaging apparatus used to perform skin imaging of individuals so that the system can perform rapid, non-invasive, point-of-care tests to determine if the individual has, or does not have, a number of specific diseases.

### BRIEF DESCRIPTION OF THE DRAWINGS

FIG. 1 is a flowchart showing an exemplary method of the present invention for creating a system and apparatus for performing rapid, accurate, non-invasive point-of-care tests for detecting various diseases and medical conditions;

FIG. 2 is a block diagram showing the components of an exemplary embodiment of the system and apparatus of the present invention for performing rapid, accurate, non-invasive point-of-care tests for detecting various diseases and medical conditions;

FIG. 3 is an exploded perspective view of an exemplary embodiment of the imaging booth component of the system and apparatus of the present invention for performing rapid, accurate, non-invasive point-of-care tests for detecting various diseases and medical conditions;

FIG. 4 shows an exemplary predetermined set of body poses for complete body imaging performed within the imaging booth shown in FIG. 3;

FIG. 5 is a picture of an exemplary embodiment of the Gas Chromatograph (GC) and an Ion Mobility Spectrometer instrument component of the system and apparatus of the present invention for performing rapid, accurate, non-invasive point-of-care tests for detecting various diseases and medical conditions;

FIG. 6 is a picture showing breath sampling of an individual using a common syringe;

FIG. 7 is a picture showing the sampled breath taken in FIG. 6 being manually injected into the exemplary embodiment of the Gas Chromatograph (GC) and an Ion Mobility Spectrometer instrument component shown in FIG. 5; and

FIG. 8 is a schematic showing the components of another exemplary embodiment of the system and apparatus of the present invention for performing rapid, accurate, non-invasive point-of-care tests for detecting various diseases and medical conditions that comprises a portable handheld screening and diseases detecting apparatus.

### DESCRIPTION OF EXEMPLARY EMBODIMENTS

The present invention combines biosensors and skin imaging in a system and apparatus for screening and detecting diseases. The method of the present invention for creating a system and apparatus for screening and detecting diseases measures VOCs emanating from the oral cavity an/or skin of individuals and also images the body of the individuals to identify patterns where the VOCs of diseased individuals match infrared waves of skin images of the diseased individuals thereby creating an infrared wave signature(s) for the disease. A filter or detector for that infrared wave signature(s) is then developed and included in a program application which processes VOC and image data obtained from individuals during testing to determine if individuals have the disease or medical condition. The present invention includes a non-invasive, accurate, rapid, real-time system and apparatus that is capable of testing individuals for various diseases and medical conditions. The system and apparatus of the present invention is particularly important in detecting rapidly spreading communicable diseases such as COVID-19 so that persons with and, in some instances more importantly without, COVID-19 can be quickly identified. The system and apparatus of the present invention provides rapid and accurate test results at point-of-sample collection without the need to contact the subjects being tested. It also provides rapid and accurate results without the need to take a sample for later analysis.

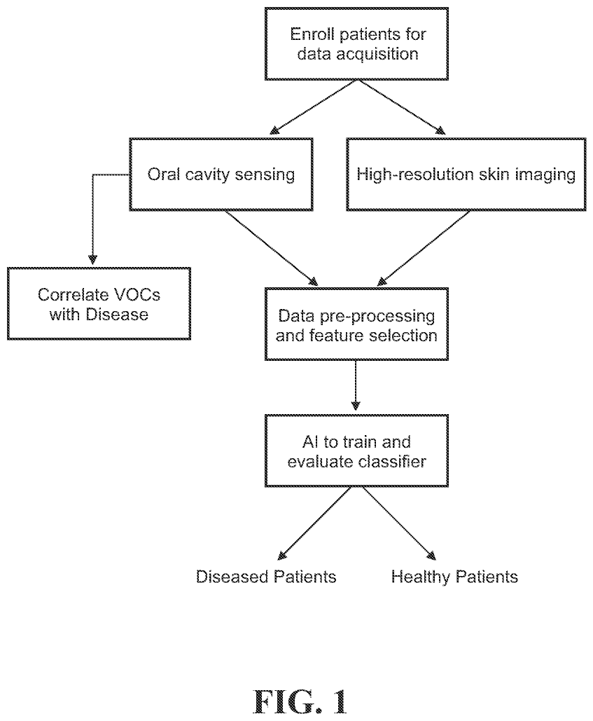

FIG. 1 is a flowchart showing an exemplary method of the present invention for creating a system and apparatus for performing rapid, accurate, non-invasive point-of-care tests for detecting various diseases and medical conditions. The system and apparatus of the present invention combines data streams from three different sources—VOCs from breath and skin and high resolution digital imaging. After enrolling patients for data acquisition, VOCs emanating from the patient's oral cavity/breath and skin are measured.

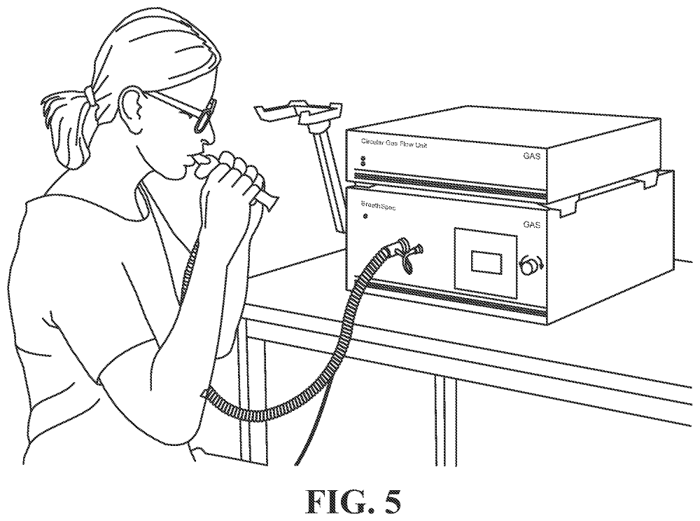

A commercial GC-IMS (gas chromatograph Ion Mobility Spectrometer) instrument, such as the G.A.S. BREATHSPEC, can be used to measure VOCs. These instruments combine a GC front end for chemical separation with a drift tube ion mobility detector. This system provides many advantages over traditional analytical techniques and more recent portable technologies (such as the electronic nose) including portability (it uses the surrounding air as the carrier gas), high sensitivity (ppb/ppt), repeatability and good chemical separation, allowing some level of chemical identification. It is a technology that found favor in the military/security sectors and that is now being used for clinical applications. In addition, the instrument is safe for users/patients and provides a result in a series of minutes. A picture of the G.A.S. BREATHSPEC is shown in FIG. 5.

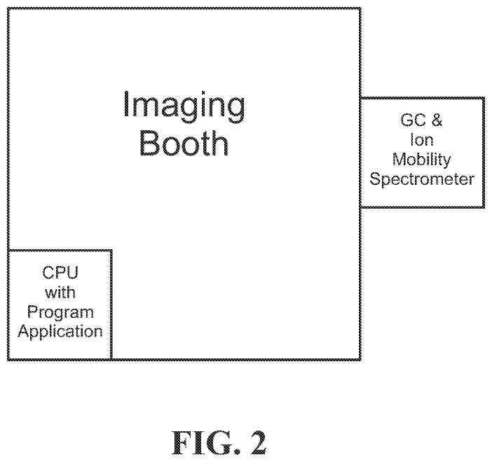

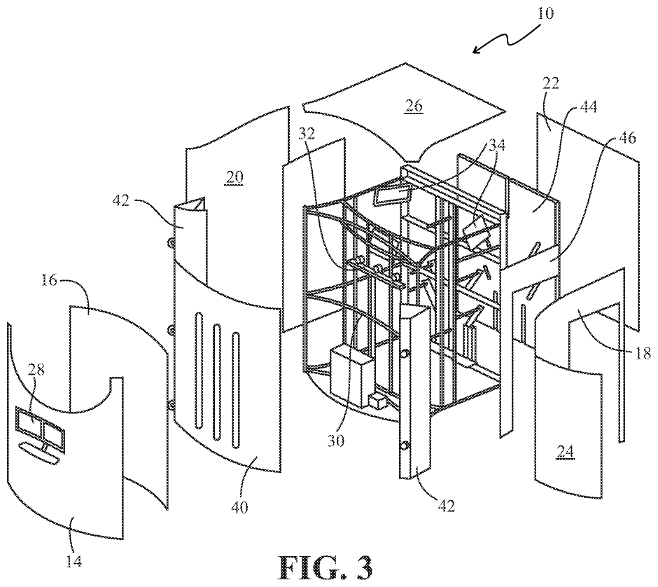

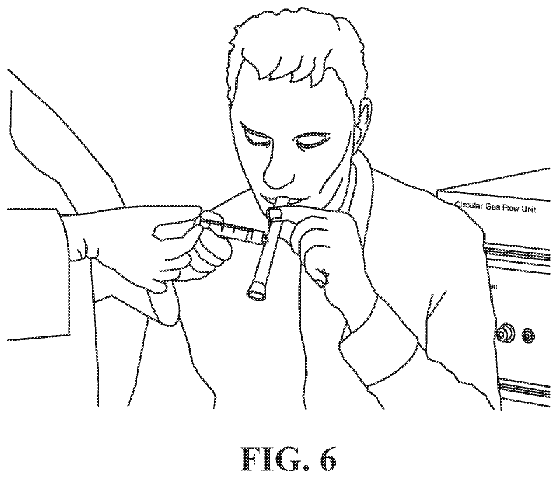

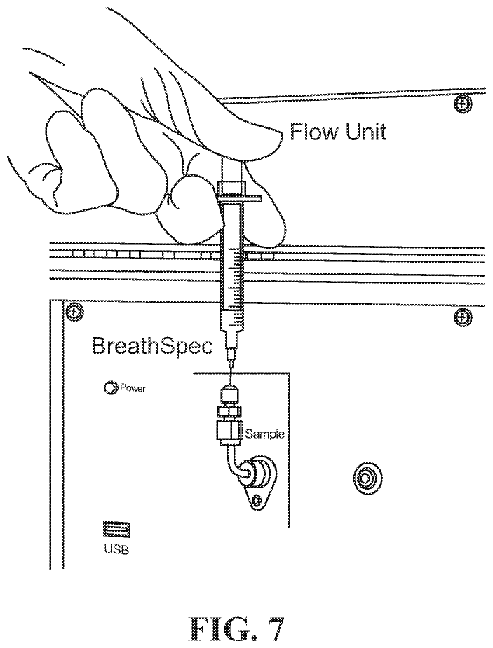

Since the GC-IMS instrument is portable and needs nothing beyond a power supply, it can run on batteries for a number of hours. FIG. 2 is a block diagram showing the components of an exemplary embodiment of the system and apparatus of the present invention for performing rapid, accurate, non-invasive point-of-care tests for detecting various diseases and medical conditions. In creating the system and apparatus for detecting diseases and medical conditions, the mouth section of the GC-IMS instrument shown in FIG. 5 can be extended into an imaging booth used for skin imaging such as the imaging booth shown in FIG. 3. The user/patient can then provide a breath sample at the same time that the user/patient is undergoing skin imaging. An alternative to extending the mouth section of the G.A.S. BREATHSPEC into the imaging booth is shown in FIGS. 6 and 7 where FIG. 6 is a picture showing breath sampling of an individual using a common syringe and FIG. 7 is a picture showing the sampled breath taken in FIG. 6 being manually injected into the exemplary embodiment of the Gas Chromatograph (GC) and an Ion Mobility Spectrometer instrument component shown in FIG. 5. Due to the high sensitivity of the GC-IMS instrument, only a single breath is needed and the capture process takes only a few seconds, with an analysis time of 5 minutes or less. For skin sampling, there is an adapter that can be purchased with the GC-IMS instrument. This was developed for the cosmetic industry to analyze the effect of skin creams etc. However, in the system and apparatus of the present invention, the adapter is used to measure VOCs emanating from the skin.

High resolution skin imaging is also performed on the patients enrolled for data acquisition. An imaging station or booth such as that shown in FIG. 3 can be utilized to perform the high resolution skin imaging. High resolution images (36.3 Mp) are captured by an array of digital cameras contained within the imaging station/booth. The imaging station/booth incorporates sensing with short wavelength IR (SWIR). The imaging station/booth may also incorporate sensing with ultraviolet based light and may further have the potential to detect emissions from the skin. One example of cameras that may be used within the imaging station/booth are those supplied by Allied Vision which offers a wide range of digital cameras with infrared sensitivity ranging from monochrome cameras with enhanced near-infrared sensitivity, to short-wave (SWIR) and long-wave (LWIR) infrared imaging devices.

The imaging station/booth includes an enclosed booth, standardized lighting, and patient-customized handlebars and footrest to allow for consistent body positioning in the most efficient space. Examples of the imaging booth/station for body/skin imaging that can be utilized as a component in the system and apparatus of the present invention are described in detail in European Patent Nos. 2846682 and 3195797, U.S. Patent Publication No. 20180125370, and U.S. Pat. No. 10,702,159. The imaging booth/station maximizes patient privacy and requires much less set-up and training than other platforms which is attributable to its booth design and highly automated imaging process.

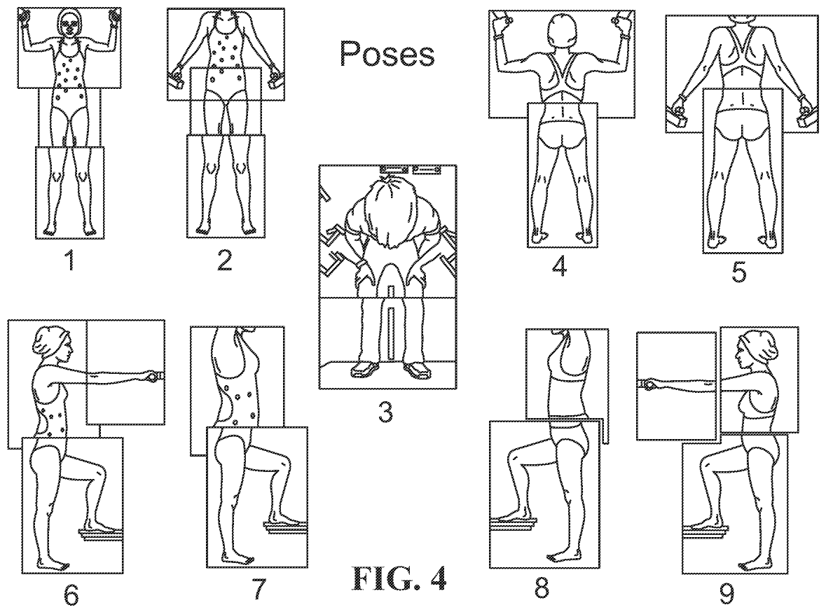

As previously discussed, FIG. 3 is an exploded perspective view of an exemplary embodiment of the imaging booth component of the system and apparatus of the present invention for performing rapid, accurate, non-invasive point-of-care tests for detecting various diseases and medical conditions. Imaging station/booth 10 includes housing having two front walls 14, 16, a first side wall 18 having an opening therein for user entry into the imaging station/booth 10, a second side wall 20, a back wall 22, a door 24 which covers the opening in side wall 18, and a top member 26. In addition, a frame 30 is located within the housing for mounting cameras 32, video displays 34, handle members 36, and a movable step 38. The housing may further include additional panel members such as front panel 40, corner panels 42 for enclosing light boxes, back panel 44, and side panel 46 all of which also function to add additional durability, strength, and support to the imaging station/booth 10. In one exemplary embodiment, the frame 30 may be aluminum and the walls and panels of the housing may be comprised of a plastic. As shown in FIG. 4, patients/individuals are guided through each of several poses by an instructional video that plays inside the booth, and operators outside the booth need only minimal training in order to operate the system. Image data is collected, uploaded and stored securely in an onsite server and back up cloud server.

After collection of VOC data and skin imaging data, the data is processed to determine/identify the infrared wave signature(s) for the disease that matches the VOCs. A filter or detector for the infrared wave signature(s) is then developed by using artificial intelligence to train and evaluate the detector. Once a filter or detector for a specific disease or medical condition is obtained, it can be incorporated into the system and apparatus of the present invention for detecting the specific disease or medical condition. A block diagram showing the components of an exemplary embodiment of the system and apparatus of the present invention for performing rapid, accurate, non-invasive point-of-care tests for detecting various diseases and medical conditions is shown in FIG. 2. The system and apparatus includes an imaging booth for skin/body imaging, a GC-IMS instrument for measuring VOCs in an individual's breath/oral cavity, and/or skin where a mouthpiece or mouth section of the GC-IMS instrument can be extended into the imaging booth, and a central processing unit with a program application for processing the VOC data, imaging data, and a disease specific filter or detector to determine if the individual has the specific disease or medical condition.

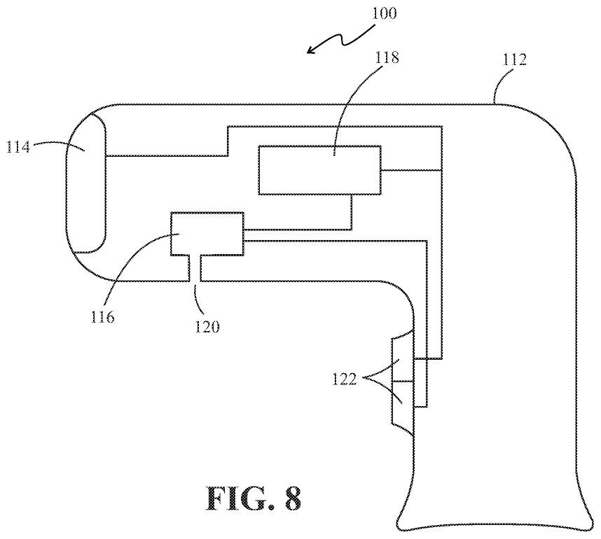

FIG. 8 is a schematic showing the components of another exemplary embodiment of the system and apparatus of the present invention for performing rapid, accurate, non-invasive point-of-care tests for detecting various diseases and medical conditions that comprises a portable handheld screening and diseases detecting apparatus 100. Portable handheld screening and disease detecting apparatus 100 includes a housing 112 that contains one or more sensors 114 for measuring wavelengths of light emanating from skin of a patient/individual, one or more sensors 116 for measuring volatile organic compounds present in a patient's/individual's breath, oral cavity, and/or skin, and a data processor 118 having a detector for one or more specific diseases or medical conditions that is capable of processing the patient's/individual's skin sensing data and volatile organic compound data to determine if the patient/individual has one or more of the specific diseases or medical conditions. The housing 112 of the portable handheld screening and disease detecting apparatus 100 may also include an inlet 120 for receiving a breath sample from the patient/individual where the inlet 120 is connected to the one or more sensors 116 for measuring volatile organic compounds. The portable handheld screening and disease detecting apparatus 100 may also include selection/activation buttons 122 for selectively activating the one or more sensors 114 for measuring wavelengths of light emanating from skin of a patient/individual, the one or more sensors 116 for measuring volatile organic compounds present in a patient's/individual's breath, oral cavity, and/or skin, and the data processor 118. The portable handheld screening and disease detecting apparatus 100 may also include a display screen on the outer surface of the housing 112 for displaying test results.

The drawings and description of exemplary embodiments of the invention herein shows various exemplary embodiments of the invention. These exemplary embodiments and modes are described in sufficient detail to enable those skilled in the art to practice the invention and are not intended to limit the scope, applicability, or configuration of the invention in any way. Rather, the following disclosure is intended to teach both the implementation of the exemplary embodiments and modes and any equivalent modes or embodiments that are known or obvious to those reasonably skilled in the art. Additionally, all included examples are non-limiting illustrations of the exemplary embodiments and modes, which similarly avail themselves to any equivalent modes or embodiments that are known or obvious to those reasonably skilled in the art.

Other combinations and/or modifications of structures, arrangements, applications, proportions, elements, materials, or components used in the practice of the instant invention, in addition to those not specifically recited, can be varied or otherwise particularly adapted to specific environments, manufacturing specifications, design parameters, or other operating requirements without departing from the scope of the instant invention and are intended to be included in this disclosure.

1. A system for performing a rapid, non-invasive, point-of-care test for detecting the presence of a specific disease or medical condition in an individual comprising:
an imaging booth capable of performing full body high resolution multispectral skin imaging of an individual;
a gas chromatograph and ion mobility spectrometer instrument capable of analyzing the individual's breath, oral cavity, and/or skin for volatile organic compounds; and
a central processing unit with a program application having a detector for a specific disease or medical condition that is capable of processing the individual's skin imaging data and volatile organic compound data to determine if the individual has the specific disease or medical condition.

2. The system of claim 1 wherein a mouthpiece for the gas chromatograph and ion mobility spectrometer instrument extends into the imaging booth so that the individual can provide a breath sample while undergoing full body skin imaging utilizing the imaging booth.

3. The system of claim 1 wherein the imaging booth includes an array of digital cameras to capture the full body high resolution multispectral skin images of the individual.

4. The system of claim 3 wherein the array of digital cameras have infrared wavelength sensitivity capable of sensing short wave infrared wavelengths.

5. The system of claim 3 wherein the array of digital cameras have ultraviolet wavelength sensitivity capable of sensing ultraviolet wavelengths.

6. The system of claim 1 wherein the program application includes a detector for a coronavirus disease that is created by comparing volatile organic compound data and skin imaging data of individuals with the coronavirus disease and volatile organic compound data and skin imaging data of individuals without the coronavirus disease to identify an infrared wave signature for the coronavirus disease.

7. A portable remote screening and disease detecting apparatus for performing a rapid, non-invasive, point-of-care test for detecting the presence of a specific disease or medical condition in an individual comprising:
one or more sensors for measuring wavelengths of light emanating from skin of an individual;
one or more sensors for measuring volatile organic compounds present in the individual's breath, oral cavity, and/or skin; and
a data processor having a detector for a specific disease or medical condition that is capable of processing the individual's skin sensing data and volatile organic compound data to determine if the individual has the specific disease or medical condition.

8. The apparatus of claim 7 further comprising an inlet for receiving a breath sample from the individual wherein the inlet is connected to the one or more sensors for measuring volatile organic compounds.

9. The apparatus of claim 7 wherein the one or more sensors for measuring wavelengths of light emanating from the individual's skin are capable of measuring short wave infrared wavelengths.

10. The apparatus of claim 7 wherein the one or more sensors for measuring wavelengths of light emanating from the individual's skin are capable of measuring ultra violet light wavelengths.

11. The apparatus of claim 7 wherein the processor includes a detector for a coronavirus disease that is created by comparing volatile organic compound data and wavelength data from skin of individuals with the coronavirus disease and volatile organic compound data and wavelength data from skin of individuals without the coronavirus disease to identify an infrared wave signature for the coronavirus disease.

12. A rapid, non-invasive, point-of-care system for gathering data related to an individual for testing for the presence of a specific disease or medical condition in the individual comprising:
an imaging booth capable of performing full body high resolution multispectral skin imaging of an individual;
a gas chromatograph and ion mobility spectrometer instrument capable of analyzing the individual's breath, oral cavity, and/or skin for volatile organic compounds; and
a central processing unit with a program application for saving the individual's skin imaging data and volatile organic compound data and sending the individual's skin imaging data and volatile organic compound data to another location for detecting the presence of a specific disease or medical condition in the individual.

13. A portable remote apparatus for gathering data related to an individual for testing for the presence of a specific disease or medical condition in the individual comprising:
one or more sensors for measuring wavelengths of light emanating from skin of an individual;
one or more sensors for measuring volatile organic compounds present ire the individual's breath, oral cavity, and/or skin; and
a data processor with a program application for saving the individual's skin imaging data and volatile organic compound data and sending the individual's skin imaging data and volatile organic compound data to another location for detecting the presence of a specific disease or medical condition in the individual.

14. A method for creating a system and apparatus for performing a rapid, non-invasive, point-of-care test for detecting the presence of a specific disease or medical condition in an individual comprising the steps of:
utilizing bio-sensing technology to measure volatile organic compounds from a breath, an oral cavity, and/or skin of a plurality of individuals with a disease having a known volatile organic compound disease biomarker and a plurality of healthy individuals without the disease;
utilizing high resolution multispectral imaging to obtain skin images of the plurality of individuals with the disease having a known volatile organic compound disease biomarker and the plurality of healthy individuals without the disease;
processing the measured volatile organic compound data and the skin imaging data to identify an infrared wave signature for the disease from the skin imaging data that correlates with volatile organic compound data for the diseased individuals; and
developing a detector for detecting the infrared wave signature.

15. The method of claim 14 further comprising the step of utilizing the infrared wave signature in a rapid, non-invasive, point-of-care apparatus to identify individuals with the disease and individuals without the disease.

16. The method of claim 15 wherein the step of utilizing the infrared wave signature in a rapid, non-invasive, point-of-care apparatus to identify individuals with the disease and individuals without the disease comprises the step of utilizing the infrared wave signature in an apparatus that includes an imaging booth for performing full body high resolution multispectral skin imaging and a gas chromatograph and ion mobility spectrometer instrument capable of analyzing an individuals' breath, oral cavity, and/or skin for volatile organic compounds.

17. The method of claim 16 wherein a mouthpiece for the gas chromatograph and ion mobility spectrometer instrument extends into the imaging booth so that the individual can provide a breath sample while undergoing full body skin imaging utilizing the imaging booth.

18. The method of claim 14 wherein the step of utilizing the infrared wave signature in a rapid, non-invasive, point-of-care apparatus to identify individuals with the disease and individuals without the disease comprises the step of utilizing the infrared wave signature in a portable remote screening and disease detecting apparatus that includes one or more sensors for measuring wavelengths of light emanating from skin of an individual and one or more sensors for measuring volatile organic compounds present in the individual's breath, oral cavity, and/or skin.

19. The method of claim 14 wherein the disease is a coronavirus disease and the detector detects the infrared wave signature for the coronavirus disease.

20. The method of claim 15 wherein the infrared wave signature is for a coronavirus disease and the rapid, non-invasive, point-of-care apparatus is used to identify individuals with the coronavirus disease and individuals without the coronavirus disease.
